(19) United States
(12) Patent Application Publication
(10) Pub. No.: US 2007/0233005 A1
(43) Pub. Date: Oct. 4, 2007

(54) Surgical fastening tool

(76) Inventors: Donald J. McMichael, Roswell, GA (US); Scott Hooser, Riverton, UT (US); Nathan V. Shirley, Herriman, UT (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/396,812

(22) Filed: Apr. 3, 2006

(51) Int. Cl. A61M 5/178

(52) U.S. Cl. 604/164.01

(57) ABSTRACT

A surgical tool is disclosed. The tool is found useful in performing percutaneous endoscopic gastrostomy and other surgical procedures requiring formation of a stoma into a body lumen. Such a tool would contain a first and second cannulae concentrically nested within one another and secured together at a distal end. An actuator is provided for selectively engaging and disengaging a retention mechanism located on that portion of the tool positioned within the body lumen. A locking mechanism is also provided to be used in conjunction with the retention mechanism for securing the cannulae to one another enabling the cannulae to be severed, and the actuator to be disposed, until the locking mechanism is disengaged upon formation and healing of an artificial stoma.

### BACKGROUND OF THE INVENTION

The present invention relates to a surgical fastening tool and, more particularly, to a tool for percutaneously placing various gastric catheters and forming artificial stomas into the gastrointestinal tract.

For example, numerous medical conditions exist in which it becomes necessary to gain percutaneous access to viscera such as the stomach or small intestines. Situations where a patient has lost the ability to swallow and will require long term nutritional support may dictate feeding directly into the stomach or jejunum. This type of feeding may be accomplished by inserting a feeding tube into the patient's stomach such that one end remains anchored in the stomach, while the other end remains external to the patient's body for connection to a nutrient source.

Feeding tubes may be inserted into a patient's stomach in a number of ways. Feeding tubes may be endoscopically placed, surgically placed through an open incision, laproscopically placed, or percutaneously placed under endoscopic, fluoroscopic or ultrasonic guidance.

Different types of feeding tubes may be placed using these procedures, examples include gastrostomy, jejunostomy or gastro-jejunostomy. These tubes may be retained in the lumen (stomach or intestine) with a variety of retention anchors. These anchoring mechanisms include: inflatable balloons, obturatable domes, fixed dome-type bumpers, or suture wings.

When placing a tube with an inflatable balloon percutaneously, it is preferred to perform a gastropexy procedure during placement. This procedure enables the physician to attach the visceral wall to the abdomen. This attachment is critical to prevent inadvertent separation and exposure of the peritoneal cavity to contamination and possible peritonitis.

The anchoring mechanism of the prior art devices typically consist of a small metal t-shaped fastener that may embed itself into the gastric or intestinal wall and ultimately lead to infection. The t-shaped fastener or t-bar is not removable and is left in the body cavity where it is allowed to pass naturally in the patient's stool. In many cases the t-bar is not passed and remains within the body cavity. Moreover, the t-bar has sharp edges which can be uncomfortable for the patient.

What is needed is a fixation device that is easy to place within an internal body cavity, allows for the formation of a stoma between the internal body cavity and the external environment, and enables the user to easily remove the fixation device when it is no longer necessary. Such a device also capable of performing the procedure itself and subsequently becoming the fixation device would be a significant improvement to the procedures currently being performed.

### SUMMARY OF THE INVENTION

In response to the foregoing problems and difficulties encountered by those of skill in the art, the present invention is directed toward a surgical fastening tool. In one aspect, the tool may have an outer cannula which has a distal end, a proximal end, and a retention mechanism. The retention mechanism is disposed proximate to the distal end and is formed at a region on the outer cannula that contains a plurality of longitudinal slits through the cannula wall. An inner cannula also having a distal end and a proximal end may be provided. The inner cannula would be slidingly disposed within the outer cannula but would be affixed to the outer cannula at the distal end of each cannula. This would serve to form a tool tip. Sliding the cannulae with respect to one another would move the tool tip axially and cause the retention mechanism to move from a disengaged to an engaged configuration.

A slidable locking mechanism may be disposed on the outer cannula. The mechanism would be capable of positioning along the outer cannula and locking the outer and inner cannulae respectively to one another so as to prevent slidable movement between the cannulae. The locking mechanism may be a multi-part device which could include a clamp base and a cap. Engagement of the cap with the clamp base would cause the locking mechanism to frictionally grip and secure the locking mechanism to the outer cannula, and the outer cannula to the inner cannula. A deformable insert may be positioned between the clamp base and cap which would serve to frictionally engage the cannulae.

An actuator having at least a first and a second position may also be provided. The first position of the actuator would be characterized in that the retention mechanism would be disengaged whereas the second position would be characterized in that the retention mechanism is engaged. The actuator may also be a multi=part component including a positioning member having a catch that engages with a separate hub. The two work in conjunction to deploy the retention mechanism.

In another aspect, the surgical tool may be adapted to perform percutaneous endoscopic gastrostomy. Such a tool may have first and second cannulae concentrically nested within one another. The cannulae may be secured together at a distal end. The distal end may be adapted to penetrate a patient's abdominal wall and gastric lumen. The tool would have an actuator for selectively engaging and disengaging a retention mechanism which is located on that portion of the tool positioned within the gastric lumen. A locking mechanism may also be provided. The locking mechanism would work in conjunction with the retention mechanism to position and secure the gastric lumen proximal to the abdominal wall. Once accomplished, the locking mechanism would secure the cannulae to one another enabling the cannulae to be severed and the actuator to be disposed. At the discretion of the physician and after formation and healing of an artificial stoma to the gastric lumen the locking mechanism may be disengaged.

In either aspect, the tool may also contain a disposable needle for percutaneously piercing tissue.

Other objects, advantages and applications of the present invention will be made clear by the following detailed description of a preferred embodiment of the invention and the accompanying drawings wherein reference numerals refer to like or equivalent structures.

### BRIEF DESCRIPTION OF THE DRAWINGS

FIG. 1 is a perspective view of one embodiment of a surgical fastening tool contemplated by the present invention;

FIG. 1a is an enlarged view of the distal end of the FIG. 1 fastening tool;

FIG. 2 is a perspective view of the various components contained in the FIG. 1 surgical fastening tool;

FIG. 3 is a perspective view of the FIG. 1 embodiment shown being introduced into a body lumen;

FIG. 4 is a perspective view of the tool in place in a body lumen after severing of a portion of the tool;

FIG. 5 is a cutaway of the proximal end of the FIG. 1 embodiment of the tool;

FIG. 6 is a perspective view of an alternative variation of the FIG. 1;

FIG. 7 is a perspective view of the distal end of the FIG. 1 tool depicting the retention mechanism in a deployed state;

FIG. 8 is a cutaway view of one embodiment of locking mechanism of the FIG. 1 embodiment; and

FIG. 9 is a cutaway view of an alternative embodiment of locking mechanism of the FIG. 1 embodiment.

### DETAILED DESCRIPTION OF THE PRESENT INVENTION

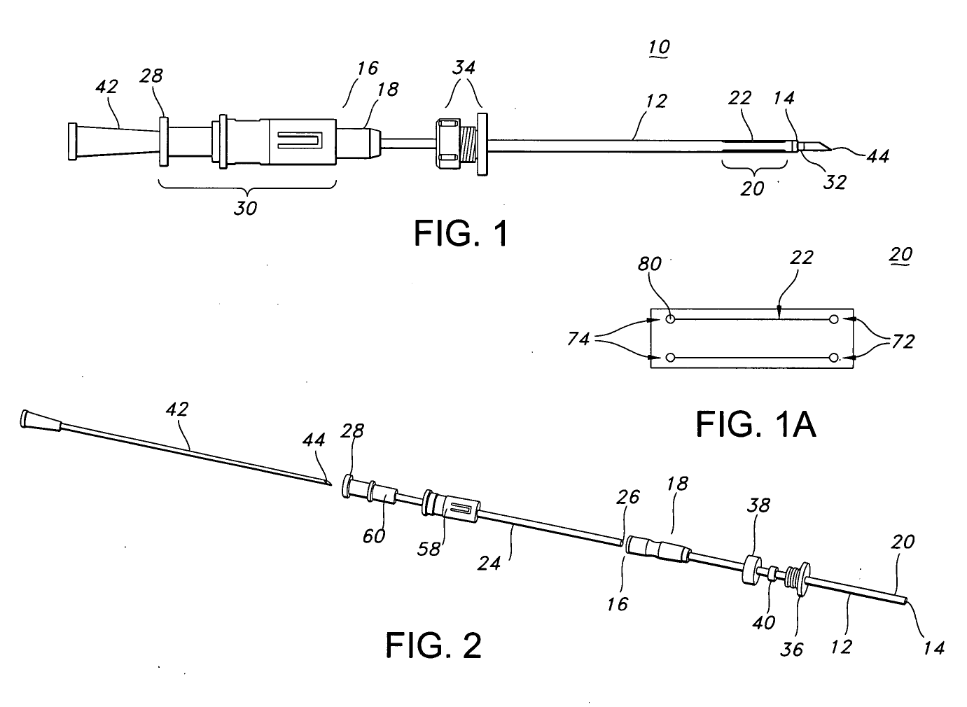

In response to the foregoing challenges that have been experienced by those of skill in the art, the present invention is directed toward a gastric fastening tool 10 for use in facilitating initial placement of enteral feeding tubes and the like as shown in the FIGs. In one embodiment, as depicted in FIG. 1 and the exploded view of FIG. 2, the tool 10 includes an outer cannula 12 having a distal end 14, a proximal end 16 terminating in a hub 18, and a region 20 that contains a plurality of longitudinal slits 22. The region 20 is disposed proximate to the distal end 14 and is shown expanded in FIG. 1a. As used herein, “distal” refers generally to the direction of the patient, while “proximal” refers generally to the direction of the user or clinician.

Looking back to FIGS. 1 and 2, it may be seen that the tool 10 also includes a second, inner cannula 24 having a distal end 26 and a proximal end 28 terminating in an actuator 30. The inner cannula 24 is slidingly disposed within the outer cannula 12, however, the two cannulae 12 and 24 are affixed to one another at their distal ends 14 and 26 respectively, to form tool tip 32. The actuator 30 engages the hub 18 and the two work in unison to move the cannulae in a desired manner as described in detail below.

A slidable locking mechanism 34 is disposed on the outer cannula 12 and is movable along its length. The locking mechanism 34 is capable of being positioned by a clinician and secured or locked onto the outer cannula 12 in such a way that the inner cannula 24 is locked to the outer cannula 12. This prevents relative movement between the outer and inner cannulae 12 and 24 respectively. The locking mechanism 34 may be a single component or a multi-part device such as depicted in the FIGs. For example, in one embodiment, the locking mechanism 34 may be a two part structure including a clamp base 36 and a cap 38 that engage one another to work synergistically to lock into a desired position. A third component, a deformable insert 40 may also be provided, each of which will be described in greater detail below.

An optional needle 42 may be provided as well which otherwise engages with the tool 10. The needle 42 is designed to slidingly engage the lumen (not depicted) through the center of the inner cannula 24 and once fully inserted, to protrude a desired distance beyond both the outer and inner cannulae 12 and 24 respectively. The needle 42 is provided with a tip 44 designed to pierce animal tissue, and may be configured with an atraumatic tip as would be understood by those of skill in the art of surgical needle design. To best understand the invention, application of the device will be described.

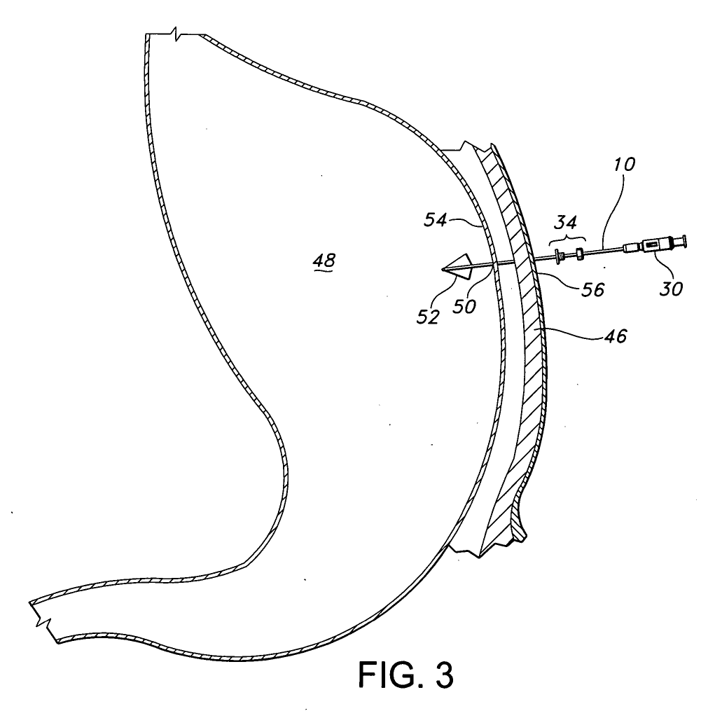

Looking now in more depth to FIG. 1, it may be seen that the needle tip 44 protrudes beyond the tool tip 32. This enables the tool 10 to be inserted into a body cavity. In one application, for example, that depicted in FIG. 3, the needle engaged within the tool is used to puncture a patient's skin and permit the tool 10 to be inserted through abdominal wall 46 into the patient's stomach lumen 48 thereby creating a stoma 50. Once the tissue has been pierced and the tool has been positioned as desired within the stomach lumen 48, the needle 42 (of FIGS. 1 and 2) may be completely withdrawn and disposed of in an appropriate manner.

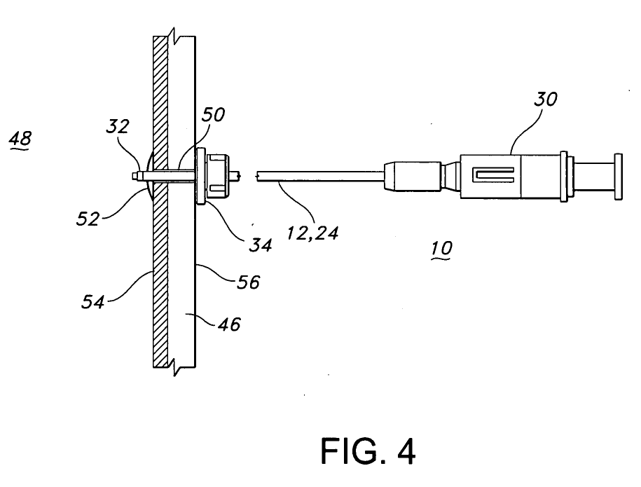

At this point the clinician would manipulate the actuator 30 to move it from a first position to a second position as shown in FIG. 3. In fact, FIG. 3 depicts the tool 10 in a condition existing at some arbitrary point in time after the clinician has manipulated the actuator 30, and as shown, this serves to deploy a retention mechanism 52. The tool 10 is then pulled back toward the clinician. This action serves to move the retention mechanism against a wall 54 of the stomach lumen 48. Continued pulling by the clinician brings the stomach lumen 48 against the abdominal wall 46, as depicted in FIG. 4, whereupon the two can be sutured to one another until formation of the stoma 50 is complete. Once the stomach lumen 48 and abdominal wall 46 are situated as desired by the clinician, the locking mechanism 34 may be slid into contact with the patient's skin at an exterior surface 56 of the abdominal wall 46. In the case of the depicted embodiment, the cap 38 may then be engaged with the clamp base 36 thereby securing the outer cannula 12 to the inner cannula 24 such that the retention mechanism 52 remains in a deployed configuration.

Once this is accomplished, the tool 10 may be cut at a point proximal to the locking mechanism 34 as shown in FIG. 4 so that both cannulae 12 and 24 are severed. This enables the gastric fastening tool 10 to be transformed into and to serve as a gastropexy device until formation of the stoma 50 is complete. Once the stoma 50 heals and is properly formed the remaining portion of the tool 10 may be removed by disengaging the locking mechanism 34. This enables the remaining portions of the cannulae 12 and 24 to slide with respect to one another once again. However, since the two cannulae are affixed to one another at the tool tip 32, the inner cannula 24 is prevented from inadvertently falling into the stomach lumen 48. Moreover, as the device is pulled from the patient by the clinician, due to the cannulae being slidable with respect to one another the retention mechanism 52 will naturally collapse thus enabling the tool to be withdrawn from the stomach lumen 48.

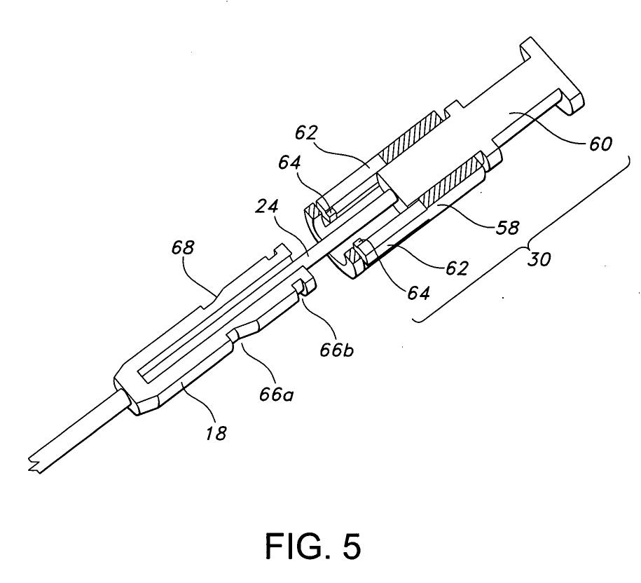

Turning now to FIG. 5 which shows the proximal end of the tool 10 in greater detail, it may be seen that in this embodiment, the actuator 30 has a distal section or positioning member 58 that is affixed to a proximal section or grip 60 which mounts to the inner cannula 24. Although these pieces 58 and 60 are described as separate pieces affixed to one another, however in alternative embodiments they may in fact comprise a single integral inseparable element. In the view depicted in FIG. 5, they are shown slightly separated along their axial length for clarity and they are also described as being separate and subsequently joined as this has been found to be more amenable to the manufacturing process. Regardless, the positioning member 58 in this embodiment has at least one catch that engages with a suitable mating device on the hub 18. In the exemplary embodiment shown in FIG. 5, there are two such catches, each configured as a finger 62 with an inwardly disposed detent 64 that engages one of two mating devices, which are configured as grooves 66. The second position, in which the detent 64 is engaged with a groove 66, specifically groove 66b is characterized by the retention mechanism 52 being in its deployed configuration. Since the retention mechanism 52 is within the patient, this serves as an important indicator to the clinician.

By having the detent 64 click or lock into place in the groove 66, the clinician may be assured that the retention mechanism 52 is properly deployed. Moreover, the actuator 30 should remain engaged so that the retention mechanism 52 is deployed until changed by the clinician. As stated earlier, the first position need not be one in which the detent 64 is engaged with a groove 66. However, in the case of the illustrated configuration, the first position may also constitute a fixed and set position such as would occur if the detent 64 were engaged with groove 66a. Additionally, a ramp 68 or other suitable design may be provided to enable the actuator to move easily from the first position. Having a specific first position may prove useful in that it would secure the tool 10 in a position in which the retention mechanism 52 is completely restrained from inadvertent deployment.

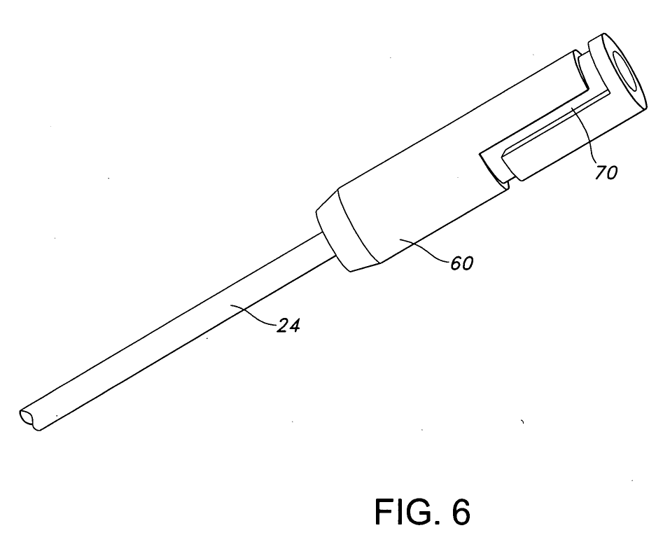

It should be understood that other catches and mating devices would work equally as well and are thus considered to form a part of this invention. For example, a pawl, a dog, a ratchet mechanism, a spring biasing means, tabs, and many other devices would be suitable for the purpose intended. In one example, as shown in FIG. 6, a groove or track 70 may be configured in either component, i.e., the positioning member 58 or the hub 18, the track 70 would form a pathway within which the detent 64, or other suitable tab or protrusion would travel. In any event, regardless of the mechanism selected, the purpose is to move the actuator 30 from some first position to a specific set and fixed second position

In embodiments depicted, pulling back on the actuator 30 in a direction away from the patient would serve to slidingly withdraw the inner cannula 24 from the outer cannula 12. Since the cannulae are connected at the tool tip 32, i.e., at the distal ends 14 and 26 of each cannula, the function that actually occurs is limited to deployment of the retention mechanism 52. The retention mechanism 52 itself, is essentially formed by the controlled collapse of the outer cannula 12 at the region 20 containing the longitudinal slits 22 and is generally referred to as a malecot-type arrangement.

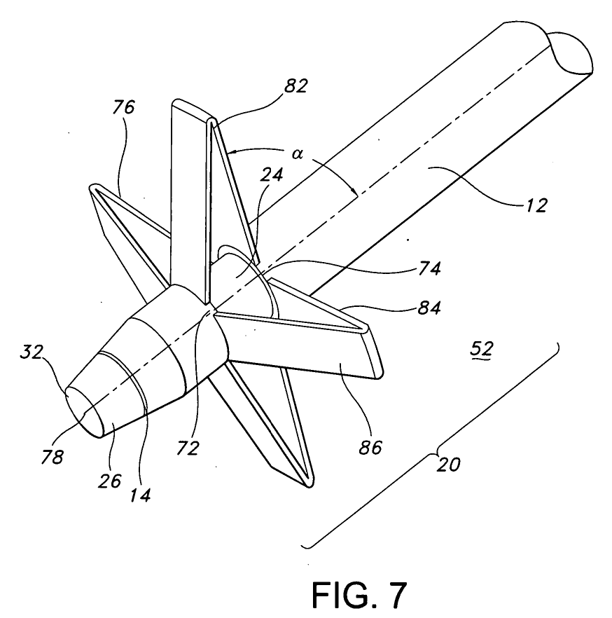

As may be seen by looking back to FIG. 1a, the region 20 comprises a plurality of the slits 22 which are radially disposed about the circumference of the outer cannula 12. The slits 22 extend axially along the length of the cannula 12 from a first end point 72 to a second end point 74. Each slit 22 may generally be of the same length and begin and end generally at the same relative positions along the cannula 12 but interspaced radially about its circumference. Looking now to FIG. 7, it may be seen that the slits 22 from FIG. 1a, form sections or wings 76. The wings 76 comprise those individual collapsible segments of the cannula 12 that lie between the slits 22 and as depicted in the FIG. 7 illustration may be seen to be generally equally spaced about a central axis 78 of the tool 10 around the circumference of cannula 12. Circular or other shaped apertures 80, best seen in FIG. 1a, may be included at the end points 72 and 74 to serve as stress relievers but are not necessary to practice the invention.

Looking still to FIG. 7, it may be envisioned that when the clinician manipulates the actuator 30 as described above, with respect to each longitudinal slit 22, the first end point 72 is being drawn toward the second end point 74. As such, the slits 22 and wings 76 are adapted to accommodate this movement. The slits 22 allow the wings 76 to bend or otherwise deform. Such deformation may be accommodated by the deformation of the entire length of each wing 76 or may be focused at one or more weakened, thinned, or necked locations 82. As shown in FIG. 7, each wing 76 would hinge outwardly at the necked location 82 such as that shown substantially mid-length along the wing 76, thus effectively collapsing the region 20 thereby effectively deploying the retention mechanism 52. This configuration should be easily understood by those skilled in the art and for all practical purposes, prevents the cannula 12 from being pulled from the stomach lumen 48.

It has been found that offsetting the necked location 82 toward the proximal end of the cannula 12 has the effect of making the wing 76 have a shorter leg 84 and a longer leg 86. This results in each wing 76 of the retention mechanism 52 having the shorter leg 84 against the stomach lumen 48. As may be seen in FIG. 7, this forces the retention mechanism 52 to form a generally right angled conical configuration. Though the angle α need not approximate 90 degrees, such a configuration would be understood by those skilled in the field of statics to be comparatively more rigid than would an embodiment wherein each leg 84 and 86 is identical in length and when actuated would lie flat against one another.

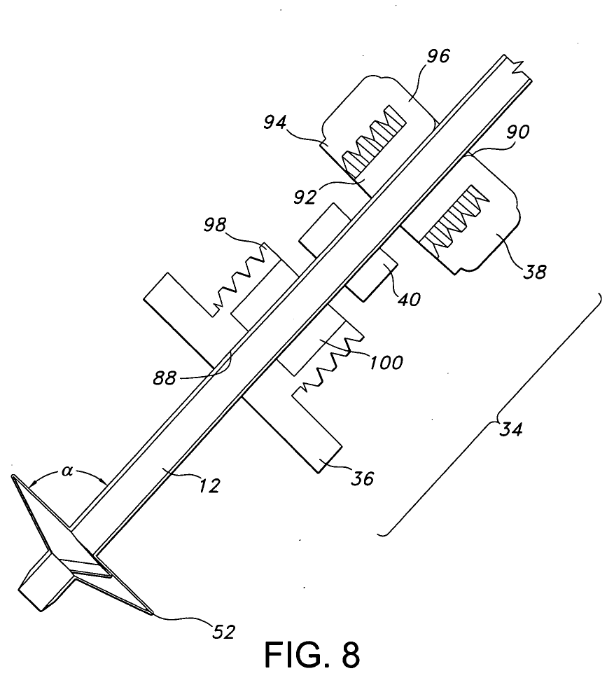

Nevertheless, once the retention mechanism 52 is engaged as described above, to lock cannulae 12 and 24 in place with respect to one another, the locking mechanism 34 is engaged. As may be seen in FIG. 8, the locking mechanism 34 may comprise two primary elements, the clamp base 36 and the cap 38. The purpose of the locking mechanism 34, as previously described, is to secure the cannulae to one another so that the retention mechanism 52 remains deployed while the stoma is formed in the patient's body. Any device capable of being secured in place to accommodate this purpose is therefore envisioned including the configuration depicted in FIG. 8.

Looking to the cutaway view of FIG. 8 in more detail, one embodiment is depicted. In this embodiment, it may be seen that the cannula 12 is situated through a central bore 88 in the clamp base 36. The cap 38 also has a central bore 90 through which the cannula 12 is also placed. The cap 38 has an inner diameter or gland 92 as well as an outer flange 94 connected by a bridge 96. The flange 94 is threaded and is adapted to engage matching threads on a collar or annulus 98 which forms an exterior surface of a packing chamber 100. The packing chamber 100 is designed to contain the deformable insert 40. As the threads on the clamp cap 38 are engaged with those on the clamp base, the gland 92 serves to deform the insert 40. By making the clamp base 36 and clamp cap 38 sufficiently more rigid than the insert 40, this deformation would serve to pinch or crush the cannulae 12 and 24 together thus securing them via friction.

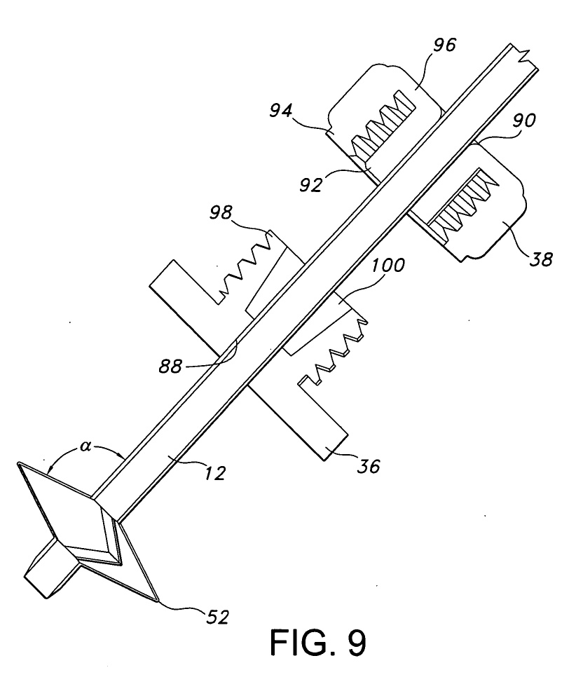

Looking to the cutaway view of FIG. 9, a different embodiment is depicted. In this embodiment there is no deformable insert. The gland 92 itself is deformable and is forced into greater contact with the cannulae due to the tapered shape of the packing chamber 100. Additionally, the angle α in this embodiment may be seen to be greater than 90 degrees as would be the case if each leg 84 and 86 on wing 76 were the same length.

In any event, once the tool is in place to the satisfaction of the clinician, the cannulae 12 and 24 may be severed, thus transforming the tool itself into a gastropexy device.

As used herein and in the claims, the term “comprising” is inclusive or open-ended and does not exclude additional unrecited elements, compositional components, or method steps.

While various patents have been incorporated herein by reference, to the extent there is any inconsistency between incorporated material and that of the written specification, the written specification shall control. In addition, while the invention has been described in detail with respect to specific embodiments thereof, it will be apparent to those skilled in the art that various alterations, modifications and other changes may be made to the invention without departing from the spirit and scope of the present invention. It is therefore intended that the claims cover all such modifications, alterations and other changes encompassed by the appended claims.

We claim:

1. A surgical fastening tool comprising:
an outer cannula having a distal end, a proximal end, and a retention mechanism disposed proximate to the distal end formed at a region having a plurality of longitudinal slits;
an inner cannula having a distal end and a proximal end, the inner cannula being slidingly disposed within the outer cannula and affixed to the outer cannula at the distal end of each cannula so as to form a tool tip wherein sliding the cannulae with respect to one another moves the tool tip axially and causes the retention mechanism to move from a disengaged to an engaged configuration;
a slidable locking mechanism disposed on the outer cannula capable of positioning along the outer cannula and locking the outer and inner cannulae respectively to one another so as to prevent slidable movement between the cannulae; and
an actuator having at least a first and a second position, the first position characterized in that the retention mechanism is disengaged and the second position characterized in that the retention mechanism is engaged.

2. The tool of claim 1 comprising a needle for percutaneously piercing tissue.

3. The tool of claim 1 wherein the retention mechanism comprises a malecot device.

4. The tool of claim 1 wherein the locking mechanism comprises a clamp base and a cap, and engagement of the cap with the clamp base causes the locking mechanism to frictionally grip and secure the locking mechanism to the outer cannula, and the outer cannula to the inner cannula.

5. The tool of claim 1 wherein the actuator comprises a positioning member, the positioning member having a catch that engages with a hub so as to deploy the retention mechanism.

6. The tool of claim 1 comprising a gastropexy device.

7. A surgical tool for performing percutaneous endoscopic gastrostomy comprising:
first and second cannulae concentrically nested within one another and secured together at a distal end adapted to penetrate a patient's abdominal wall and gastric lumen,
an actuator for selectively engaging and disengaging a retention mechanism located on that portion of the tool positioned within the gastric lumen;
a locking mechanism which in conjunction with the retention mechanism, positions and secures the gastric lumen proximal to the abdominal wall and secures the cannulae to one another enabling the cannulae to be severed, and the actuator to be disposed, until the locking mechanism is disengaged upon formation and healing of an artificial stoma to the gastric lumen.

8. The surgical tool of claim 7 comprising a disposable needle for piercing the abdominal wall.
